(19) United States
(12) Patent Application Publication
(10) Pub. No.: US 2004/0138198 A1
(43) Pub. Date: Jul. 15, 2004

(54) Vasectomy procedure and related kit

(76) Inventors: David C. Sokal, Durham, NC (US); Marc Goldstein, New York, NY (US)

Correspondence Address: Richard S Faust, Suite 204, 8384 Six Forks Road, Raleigh, NC, 27615, US

(21) Appl. No.: 10/471,950

(22) PCT Filed: Apr. 17, 2002
(86) PCT No.: PCT/US02/12017

(30) Foreign Application Priority Data
Apr. 18, 2001 (US) 60284622

(51) Int. Cl. A61K 31/554; A61K 31/5415

(52) U.S. Cl. 514/211.07; 514/224.8

(57) ABSTRACT

A vasectomy procedure is performed using a vas irrigation solution having a spermicidal agent that (a) acts to reduce the viability of downstream sperm following a vasectomy procedure and (b) has been established as safe and non-toxic in humans when administered by intravenous or other routes of injection for the treatment of other conditions. Preferred spermicidal agents are methylene blue and diltiazem. A kit having a pair of single-use, pre-filled injection devices is also disclosed.

### FIELD OF THE INVENTION

The invention relates to vasectomy procedures. More particularly, the invention relates to vasectomy procedures that incorporate vas irrigation. The invention also relates to kits including pre-filled injection devices containing vas irrigation solution.

### BACKGROUND OF THE INVENTION

The traditional vasectomy procedure does not lead to immediate sterility. There are several hundred million live sperm downstream of where the vas is cut. The term “downstream” sperm refers to the sperm that are already in the distal or prostatic end of the vas deferens, on the downstream side of the cut made at the time of a vasectomy. Most surgeons warn men undergoing a vasectomy not to rely on their vasectomy for contraception until after a semen analysis at 10 to 12 weeks. In developing countries where semen testing may not be available, men are commonly advised to wait until after 12 weeks or 20 ejaculations before relying on the vasectomy for contraception.

Anecdotal data in the literature have suggested that many of the pregnancies occurring shortly after vasectomy are due to the residual downstream sperm. Some men may not understand the need for a waiting period, or they may use a contraceptive method, such as a condom, which fails. Data from a recent survey among a sample of United States urologists suggests that about 50% of pregnancies after vasectomy are due to the downstream sperm. The rate of such pregnancies is likely to be higher in developing countries, because the educational and literacy levels of men are much lower.

A small number of urologists have used and recommend a technique called “vas irrigation” which can reduce the risk of pregnancy in the post-vasectomy period. With this technique, the surgeon rinses out downstream sperm at the same time that he does the vasectomy. Articles in the literature have described methods for vas irrigation using a standard needle and syringe with various solutions, such as sterile water, sterile saline or xylocalne. Xylocalne may be used because a solution of xylocalne is the same solution used for local anesthesia before the procedure. Some physicians have used vas irrigation employing spermicidal or sperm-immobilizing agents such as euflavine, chlorhexidine, nitrofurantoin, phenyl mercuric nitrate and nitrofurazone. For various reasons specific to each agent, none of these have been generally accepted.

Given the lack of an FDA approved product, and relatively few published studies, urologists have been reluctant to use a vas irrigation technique. Some urologists have said that they do not use the technique because they do not know whether or not there might be an increased risk of adverse events or infections subsequent to the procedure.

### SUMMARY OF THE INVENTION

The present invention, in one aspect, may be defined as the use of a vas irrigation solution containing a spermicidal agent that (a) acts to reduce the viability of downstream sperm following a vasectomy procedure and (b) has been established as safe and non-toxic in humans when administered by intravenous or other routes of injection for the treatment of other conditions.

In another aspect, the invention may be defined as a vasectomy procedure including the step of vas irrigation utilizing an irrigation solution containing a spermicidal agent that (a) acts to reduce the viability of downstream sperm following a vasectomy procedure and (b) has been established as safe and non-toxic in humans when administered by intravenous or other routes of injection for the treatment of other conditions.

In yet another aspect, the invention may be defined as a kit for use in a vas irrigation associated with a vasectomy procedure. The kit comprises at least one injection device and a pre-measured amount of a vas irrigation solution pre-filled in the injection device. The solution contains a spermicidal agent that (a) acts to reduce the viability of downstream sperm following a vasectomy procedure and (b) has been established as safe and non-toxic in humans when administered by intravenous or other routes of injection for the treatment of other conditions.

In another aspect, the invention may be defined as a vas irrigation solution containing a spermicidal agent that (a) acts to reduce the viability of downstream sperm following a vasectomy procedure and (b) has been established as safe and non-toxic in humans when administered by intravenous or other routes of injection for the treatment of other conditions.

In preferred embodiments, the spermicidal agents may be a calcium channel blocker or methylene blue.

In preferred embodiments, the spermicidal agents may be methylene blue or diltiazem.

In preferred embodiments, the spermicidal agent and its concentration may be selected so that the spermicidal agent acts over a period of about 15 minutes or more to reduce the viability of downstream sperm.

### DETAILED DESCRIPTION OF THE INVENTION

According to the present invention, at the time of a vasectomy procedure a relatively small volume of a vas irrigation solution containing a spermicidal agent is injected into each prostatic side of the vas. Preferred spermicidal agents are (1) a calcium channel blocker, and (2) methylene blue. More preferred spermicidal agents are (I) diltiazem, a calcium channel blocker, and (2) methylene blue. The two above spermicidal agents have been established as safe and non-toxic in humans when administered by intravenous or other routes of injection for the treatment of other conditions, and are marketed in the United States for these uses. These agents are available in sterile preparations for intravenous use.

In preferred embodiments, the vas irrigation solutions containing spermicidal agents are used in relatively small volumes, for example, on the order of 1 to 10 ml per injection, preferably 1 to 5 ml per injection and more preferably 3 ml or less per injection. Preferably, the amount of spermicidal agent delivered to each side is 1 mg to 10 mg, preferably 2.5 mg. Also, preferably, the spermicidal agents are used at low concentrations that act over relatively long time periods, for example, over 15 minutes to 2 hours, or even more. In this regard, it will be appreciated that spermicidal activity in typical contraceptive uses such as the use of N-9 gel in human vaginal contraceptives such as gels or sponge devices usually must be relatively fast acting, for example, less than 30 seconds. Such rapidly acting agents are usually more irritating than agents with slower action. Such rapid action is not needed in the case of vas irrigation at the time of a vasectomy.

According to certain embodiments of the invention, vas irrigation is carried out using a safe, inexpensive, easy-to-use injection device with a blunt needle having means for delivering the predetermined amount of spermicidal agent. Most preferably, the injection device is a ready-to-use, disposable device that can be used only one time. While blunt metallic needles are inexpensive and commonly produced for uses such as this, a plastic introducer tip or a piece of plastic tubing may also be used, as the tip of the device does not necessarily need to penetrate the vas. In most vasectomy procedures, the lumen of the vas is already exposed by the surgeon, so that this device would only need to guide the flow of the vas irrigation solution from the container into the exposed lumen of the vas. In what follows, the term “introducer tip” is used to denote either a blunt metallic needle or a plastic cannula or similar feature of the device.

The invention may be carried out with a single-use kit that includes a pair of injection devices as described above and a pre-measured amount of vas irrigation solution containing spermicidal agent appropriate for the procedure.

### EXAMPLE I

During a vasectomy procedure, a kit is used to inject a predetermined quantity of a vas irrigation solution containing spermicidal agent into each prostatic side of the vas. The kit includes two single-use, pre-filled injection devices such as the Uniject™ injection device manufactured by Becton Dickinson Pharmaceutical Systems of Franklin Lakes, N.J., USA. Injection devices of this type are described in U.S. Pat. Nos. 4,955,871 and 5,222,948, the contents of which are incorporated herein by reference. The mentioned injection device should have a blunt needle, for example a 22 gauge needle, and a reservoir holding the vas irrigation solution containing spermicidal agent, in this example approximately one ml of undiluted or diluted sterile methylene blue solution. Methylene blue is a marketed product in the United States that has been used for diagnostic purpose in both the male and female genital tract at concentrations ranging from about 0.1% to 1%. Methylene blue may also be given by intravenous administration for the treatment of certain poisonings. Methylene blue may be used in connection with this invention in similar concentrations, for example, from 1 mg/ml to 10 mg/ml, and more preferably about 2.5 mg/ml. Thus, with an injected volume of one ml per vas, at the stated concentrations the amount of methylene blue delivered to each side ranges from 1 to 10 mg, with about 2.5 mg being one preferred dosage.

### EXAMPLE II

A procedure and kit as described in Example I is used except that the spermicidal agent in the vas irrigation solution is diltiazem, an agent that is currently available and FDA approved as a sterile formulation for IV administration for the treatment of cardiovascular disorders. It is a drug in the class of calcium channel blockers. One ml of this vas irrigation solution is injected at a concentration of 1 mg/ml to 10 mg/ml, preferably, about 2.5 to 5 mg/ml. Thus, with an injected volume of one ml per vas, at the stated concentrations the amount of diltiazem delivered to each side ranges from 1 to 10 mg, with about 2.5 mg being one preferred dosage.

### Other Spermicidal Agents and Other Uses of the Invention

Examples I and II, above, identify two preferred spermicidal agents for use in vas irrigation solutions of the invention and for incorporation into kits of the invention. Spermicidal agents appropriate for use with the invention include agents that (a) act to reduce the viability of downstream sperm following a vasectomy procedure and (b) have been established as safe and non-toxic in humans when administered by intravenous or other routes of injection for the treatment of other conditions. It is preferred that the vas irrigation solutions contain spermicidal agents with appropriate spermicidal activity and at such concentrations that they act over a time period of about 15 minutes or more.

It will be appreciated that vas irrigation solutions of the invention may include more than one spermicidal agent. Also, vas irrigation solutions containing different spermicidal agents may be administered sequentially. Furthermore, the spermicidal agent may be packaged in freeze dried form and dissolved in a solvent prior to use. To this end, the freeze dried spermicidal agent may reside in one compartment of a delivery device while the solvent resides in another compartment, with the two being mixed together prior to use.

### Test Results

A dose ranging non-GLP study was conducted in rats to determine the local toxic effect of diltiazem on vasa deferentia. Injection of diltiazem, inside the vas, at a dose of 0.2 mg/kg body weight, which is the approximate intended dose for humans, did not cause any changes in the histology of the vas. Even at a higher dose of 2.0 mg/kg, there was no effect on the vas. The general health of rats was also not affected at these doses over a period of two days. There was no mortality of rats following injection with a much higher dose of 50 mg/kg.

In another non-GLP study, the in vivo effect of diltiazem on motility and viability of sperm in vas was observed. Injection of diltiazem, at a dose of 0.2 mg/kg body weight, significantly increased the percentage of non-viable sperm to 92%, compared to only 35% for saline injected controls.

### Use of Invention in Conjunction With Other Types of Vasectomy Procedures

The vas irrigation solutions of the invention may be used in combination with other devices or drugs for improving the effectiveness of vasectomy. For example, devices disclosed in other patents, for example Thompson, U.S. Pat. No. 5,471,997; Zaneveld, U.S. Pat. No. 4,512,342; and Guha, U.S. Pat. No. 5,488,075, call for insertion of devices or drugs that can later be removed to provide, in effect, a reversible vasectomy. Vas irrigation solutions containing diltiazem, methylene blue or other suitable spermicidal agent may be used in conjunction with these methods to provide immediate infertility, so that a waiting period would not be needed after the insertion of these devices or drugs.

While the present invention has been described in connection with certain embodiments, it will be appreciated that modifications may be made without departing from the true spirit and scope of the invention.

1. The use of a vas irrigation solution containing a spermicidal agent that (a) acts to reduce the viability of downstream sperm following a vasectomy procedure and (b) has been established as safe and non-toxic in humans when administered by intravenous or other routes of injection for the treatment of other conditions.

2. The use of claim 1, wherein the spermicidal agent in the vas irrigation solution includes methylene blue.

3. The use of claim 2, wherein methylene blue is present in the solution administered in the vas irrigation in an amount of about 1 to 10 mg per side.

4. The use of claim 1, wherein the spermicidal agent in the vas irrigation solution includes a calcium channel blocker.

5. The use of claim 1, wherein the spermicidal agent in the vas irrigation solution includes diltiazem.

6. The use of claim 5, wherein diltiazem is present in the solution administered in the vas irrigation in an amount of about 1 to 10 mg per side.

7. The use of claim 5, wherein diltiazem is present in the solution administered in the vas irrigation in a concentration of about 1 to 10 mg/ml.

8. The use of claim 1, wherein the spermicidal agent acts over a time period of about 15 minutes or more to reduce the viability of downstream sperm.

9. The use of claim 8, wherein the spermicidal agent includes methylene blue.

10. The use of claim 8, wherein the spermicidal agent includes diltiazem.

11. A vasectomy procedure including the step of vas irrigation utilizing an irrigation solution containing a spermicidal agent that (a) acts to reduce the viability of downstream sperm following a vasectomy procedure and (b) has been established as safe and non-toxic in humans when administered by intravenous or other routes of injection for the treatment of other conditions.

12. The procedure of claim 1 1, wherein the vas irrigation step is carried out on each vas utilizing an injection device having an introducer tip such as a blunt needle.

13. The procedure of claim 12, wherein the introducer tip is contained in a single-use injection device pre-filled with the irrigation solution for a single vas side.

14. The procedure of claim 13, carried out with a kit containing a pair of single-use injection devices, with each injection device being pre-filled with the irrigation solution for a single vas side.

15. The procedure of claim 1 1, wherein the spermicidal agent in the vas irrigation solution includes methylene blue.

16. The procedure of claim 15, wherein methylene blue is present in the solution administered in the vas irrigation in a concentration in the range from about 1 to 10 mg/ml.

17. The procedure of claim 11, wherein the spermicidal agent in the vas irrigation solution includes a calcium channel blocker.

18. The procedure of claim 11, wherein the spermicidal agent in the vas irrigation solution contains diltiazem.

19. The procedure of claim 11, wherein diltiazem is present in the vas irrigation solution in a concentration in the range from about 1 to 10 mg/ml, and is administered in a total amount of about 1 to 10 mg.

20. The procedure of claim 11, wherein the vas irrigation solution is administered to each side in a volume on the order of 1 to 10 ml.

21. The procedure of claim 11, wherein the vas irrigation solution is administered to each side in a volume on the order of 1 to 5 ml.

22. The procedure of claim 11, wherein the vasectomy procedure is a reversible vasectomy procedure.

23. The procedure of claim 11, wherein the spermicidal agent acts over a time period of about 15 minutes or more to reduce the viability of downstream sperm following a vasectomy procedure.

24. The procedure of claim 23, wherein the spermicidal agent includes methylene blue.

25. The procedure of claim 23, wherein the spermicidal agent includes diltiazem.

26. A kit for use in a vas irrigation associated with a vasectomy procedure, the kit comprising:
at least one injection device; and
a pre-measured amount of a vas irrigation solution pre-filled in the injection device, said solution containing a spermicidal agent that (a) acts to reduce the viability of downstream sperm following a vasectomy procedure and (b) has been established as safe and non-toxic in humans when administered by intravenous or other routes of injection for the treatment of other conditions.

27. The kit of claim 26, wherein the kit contains two single-use injection devices, each device being pre-filled with a dosage of vas irrigation solution appropriate for a single vas.

28. The kit of claim 26, wherein the spermicidal agent acts over a time period of about 15 minutes or more to reduce the viability of downstream sperm following a vasectomy procedure.

29. The kit of claim 28, wherein the spermicidal agent includes methylene blue.

30. The kit of claim 28, wherein the spermicidal agent includes diltiazem.

31. A vas irrigation solution containing a spermicidal agent that (a) acts to reduce the viability of downstream sperm following a vasectomy procedure and (b) has been established as safe and non-toxic in humans when administered by intravenous or other routes of injection for the treatment of other conditions.

32. The vas irrigation solution of claim 31, wherein the spermicidal agent in the vas irrigation solution includes methylene blue.

33. The vas irrigation solution of claim 32, wherein methylene blue is present in the solution administered in the vas irrigation in an amount of about 1 to 10 mg per side.

34. The vas irrigation solution of claim 31, wherein the spermicidal agent in the vas irrigation solution includes a calcium channel blocker.

35. The vas irrigation solution of claim 31, wherein the spermicidal agent in the vas irrigation solution includes diltiazem.

36. The vas irrigation solution of claim 35, wherein diltiazem is present in the solution administered in the vas irrigation in an amount of about 1 to 10 mg per side.

37. The vas irrigation solution of claim 35, wherein diltiazem is present in the solution administered in the vas irrigation in a concentration of about 1 to 10 mg/ml.

38. The vas irrigation solution of claim 31, wherein the spermicidal agent acts over a time period of about 15 minutes or more to reduce the viability of downstream sperm following a vasectomy procedure.

39. The vas irrigation solution of claim 38, wherein the spermicidal agent includes methylene blue.

40. The vas irrigation solution of claim 38, wherein the spermicidal agent includes diltiazem.

41. A vas irrigation solution containing diltiazem as a spermicidal agent.

42. The vas irrigation solution of claim 41, wherein diltiazem is present in the solution in a concentration of about 1 to 10 mg/ml.

43. The vas irrigation solution of claim 41, packaged in dosage units for injection at about 1 to 10 mg diltiazem per vas side.

44. A vas irrigation solution containing methylene blue as a spermicidal agent.

45. The vas irrigation solution of claim 44, wherein methylene blue is present in the solution in a concentration of about 1 to 10 mg/ml.

46. The vas irrigation solution of claim 44, packaged in dosage units for injection at about 1 to 10 mg methylene blue per vas side.

47. A vas irrigation solution containing an effective amount of a spermicidal agent that, when injected into one vas side at a dosage of about 1 to 5 ml, acts to reduce the viability of downstream sperm following a vasectomy procedure over a time period of about 15 minutes or more.
